(19) United States
(12) Patent Application Publication
(10) Pub. No.: US 2018/0330815 A1
(43) Pub. Date: Nov. 15, 2018

(54) DYNAMICALLY-ADAPTIVE OCCUPANT MONITORING AND INTERACTION SYSTEMS FOR HEALTH CARE FACILITIES

(71) Applicant: Ouva, LLC, San Francisco, CA (US)

(72) Inventors: Dogan Demir, San Francisco, CA (US); Metin Nacar, San Francisco, CA (US)

(21) Appl. No.: 15/979,458

(22) Filed: May 14, 2018

Related U.S. Application Data
(60) Provisional application No. 62/505,966, filed on May 14, 2017.

(51) Int. Cl. G16H 40/20; G08B 21/24; G08B 7/06; G06K 9/00; G08B 21/18; G06Q 10/10

(52) U.S. Cl. CPC G16H 40/20; G08B 21/245; G08B 7/066; G06Q 10/1095; G08B 21/182; G06K 9/00302; G06K 9/00778; G06K 9/00288

(57) ABSTRACT

Systems and methods for monitoring and interacting with occupants of a health care facility may improve services and patient experience. Sensors, such as video camera sensors, are distributed within a health care facility, transmitting video and other information to a central processing hub in order to identify behavioral and physical events, such as patients seeking wayfinding assistance, patients queueing at a reception desk, patient wait times and experiences, and staff interaction with hand hygiene stations. Reports and notifications may be transmitted to staff in order to proactively address, e.g., patient dissatisfaction, patient deterioration, and staff compliance with processes.

### BACKGROUND

The present invention relates in general to health care services, and more specifically, to systems and methods for dynamically improving the experience of patients and other occupants in a health care facility.

Continuing research and investment in medical devices, pharmaceuticals and surgical techniques have resulted in rapid and continual improvements in the ability of health care practitioners to achieve successful patient outcomes. However, patient outcomes, as well as patient satisfaction with health care services delivered to them, are also heavily impacted by the quality of experience that a patient has in interacting with the health care facility at which they are being treated. Health care facility features that directly affect a patient's experience may include factors such as how a physical environment is built, the workflow of staff, quality of the environment and safety and security of the people that occupy the space.

Traditionally, improvements to health care facilities tend to be very capital and time intensive. Behavioral metrics and techniques for improving facilities are sometimes gathered through extensive surveys or post-occupancy studies. However, such efforts are typically very costly and time consuming. Data acquired tends to be very one-dimensional (e.g. limited to written surveys). Data acquired is also typically collected after-the-fact, concerning a patient's historical interactions, and therefore may be colored by the patient's memory and subsequent experiences. Even if such efforts yield actionable insight, many hospitals and other facilities will then lack budget to implement the facility improvements suggested by the studies.

### SUMMARY

Using a database, intelligence engine, interfaces and sensors, a health care facility can measure and respond to events that affect the quality of a healthcare environment. A system may be capable of using a sensor to identify behavioral and physical events that are related to the patient experience, staff activities and equipment, and combine these events to engage patients in an interactive conversation or alert the staff to improve the condition. Various combinations of large-scale displays, audio content, ambient lighting, and the like may be utilized to create highly immersive experiences for facility occupants. The system, outfitted with a centralized intelligence engine, collects insights from each interaction, contemporaneously with the interaction, in order to optimize the responses and effectiveness of content displayed and actions taken by staff. Visual awareness and/or patient health sensors enable direct observation of facility occupants and their reactions. By using a software-defined patient interface, the system can quickly prototype and improve the physical experience (such as wayfinding cues) for each person individually and without the need for major changes to the infrastructure. The centralized intelligence engine can then adapt the learned approaches to multiple facilities.

For example, in accordance with one aspect, a system for monitoring and interacting with occupants of a health care facility is provided. One or more video sensors are distributed throughout a health care facility at known locations. Each video sensor streams information about observed content to a processing hub. The processing hub includes application logic implementing a video content analysis module, which determines, for each of one or more individuals, an individual identity and a state or activity associated with the individual. One or more facility staff terminals receive notification of identity, location and state or activity associated with one or more of the individuals. For example, the video content analysis module may be configured to recognize staff interaction with hand hygiene stations, enabling reporting on hand hygiene compliance and/or real-time queues to prompt staff hand hygiene compliance. As another example, the video content analysis module may include application logic for tracking patient emotional state, such that facility staff computer terminals may be notified of dissatisfied individuals or individuals with deteriorating conditions. As another example, the video content analysis module may include application logic for tracking patient wait time in a waiting room, such that facility staff may be notified when waiting time exceeds desired levels.

In accordance with another aspect, a method for personalized wayfinding in a health care facility is provided. A plurality of wayfinding stations are distributed at known locations within a health care facility. The wayfinding stations may include a digital display screen, a video camera sensor, and a compute engine. A patient is identified at a first wayfinding station, such as by performing facial recognition on a captured image that is transmitted to a centralized facility intelligence server and/or by querying the patient, e.g. using a chat bot interface. A central data repository is queried to identify an intended destination associated with the identified patient. The digital display screen may then display wayfinding instructions directing the patient from the known location of the first wayfinding station, to the intended destination. When the patient arrives at the intended destination, a destination wayfinding station may report the arrival to a central intelligence engine server. The central server may then determine actual transit times for the patient. Facility staff may be notified of a divergence between actual transit times for the patient and expected transit times. Such notifications and reporting may be helpful in optimizing wayfinding directions and thereby improving the patient experience.

In accordance with another aspect, methods and systems are provided for monitoring patient satisfaction in a health care facility waiting area. One or more patients are identified in the waiting area by transmitting a plurality of images, each captured at a known time, from one or more video cameras directed towards the waiting area, to a processing hub implementing application logic comprising an image recognition component. The image recognition component can be applied by the processing hub to uniquely identify each of one or more patients in the images. The processing hub may then track a waiting duration of time during which each unique patient is present in the waiting area. If a patient's waiting duration exceeds a threshold level, staff may be notified. In some cases, the threshold waiting duration may be predetermined. In some cases, the waiting duration threshold level is determined relative to a patient's appointment time, which appointment time can be determined by querying a patient scheduling service. The processing hub may also apply an emotion evaluator image processing module to captured images of the waiting area. In the event that one or more patients is illustrating signs of an unsatisfactory emotional stage (which may be determined, e.g., by facial expression recognition), a notification may be transmitted to a computing device associated with facility staff, identifying the one or more patients illustrating signs of an unsatisfactory emotional state. This may enable staff to promptly address dissatisfied patients, such as by providing updated wait time estimates and/or verifying whether the patient's condition is deteriorating. The processing hub may additionally or alternatively apply image analysis component to images captured from a video camera directed towards a reception station, to determine the number of individuals queued at the reception station. The processing hub may transmit a notification to an electronic device associated with facility staff, in response to determination that the number of people waiting at the reception desk exceeds a directed threshold. This information may be utilized to, e.g., allow facility staff to quickly redeploy resources or otherwise address unexpectedly high check in times.

In accordance with another aspect, a method for monitoring hand hygiene compliance in a health care facility is provided. Examination rooms and other areas of a health care facility may include video camera sensors. A video feed from a video camera sensor installed in an examination room and directed towards a hand hygiene station, may be transmitted to a processing hub. A facility staff member may be identified upon entry to the examination room, such as via facial recognition from the video camera feed and/or querying identification from a separate identification server (e.g. using RFID or swipe card IDs). Content from the video feed may be applied to an image analysis component implemented by processing hub application logic, the image analysis component configured to detect staff member interaction with the hand hygiene station and generate hand hygiene compliance logs. The hand hygiene compliance logs may then be used to generate hand hygiene compliance reporting. In the absence of detecting staff member interaction with the hand hygiene station, the processing hub may initiate the display of a compliance reminder on an examination room digital display.

These and other aspects will become apparent in light of the drawings and other disclosure provided herein.

### BRIEF DESCRIPTION OF THE DRAWINGS

FIG. 1A is a schematic block diagram of a health care facility monitoring and optimization platform, in accordance with one embodiment.

FIG. 1B is a perspective view of a portion of a health care facility implementing a monitoring and optimization platform.

FIG. 1C is a schematic block diagram illustrating data flows within a health care monitoring and optimization platform.

FIG. 2A is a process diagram for personalized wayfinding.

FIG. 2B is a process diagram for wayfinding optimization.

FIG. 3 is a process diagram for patient state monitoring and optimization.

FIG. 4 is a schematic block diagram of an integrated sensor and interface.

FIG. 5 is a process for ameliorating patient fall risk.

FIG. 6 is a perspective view of a health care facility implementing a system for patient fall risk monitoring.

FIG. 7 is a schematic block diagram of a waiting room monitoring system.

FIG. 8 is a schematic perspective view of an exam room configured for hand hygiene compliance monitoring.

FIG. 9 is a schematic block diagram of a multi-facility platform, in accordance with a first embodiment.

FIG. 10 is a schematic block diagram of a multi-facility platform, in accordance with a second embodiment.

### DETAILED DESCRIPTION OF THE DRAWINGS

While this invention is susceptible to embodiment in many different forms, there are shown in the drawings and will be described in detail herein several specific embodiments, with the understanding that the present disclosure is to be considered as an exemplification of the principles of the invention and is not intended to limit the invention to the embodiments illustrated.

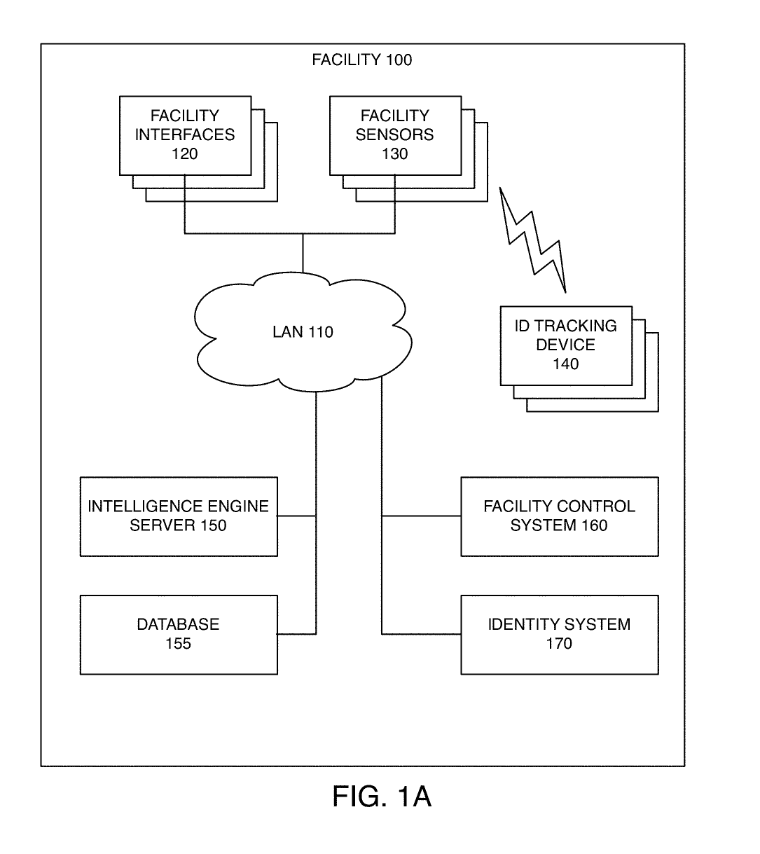

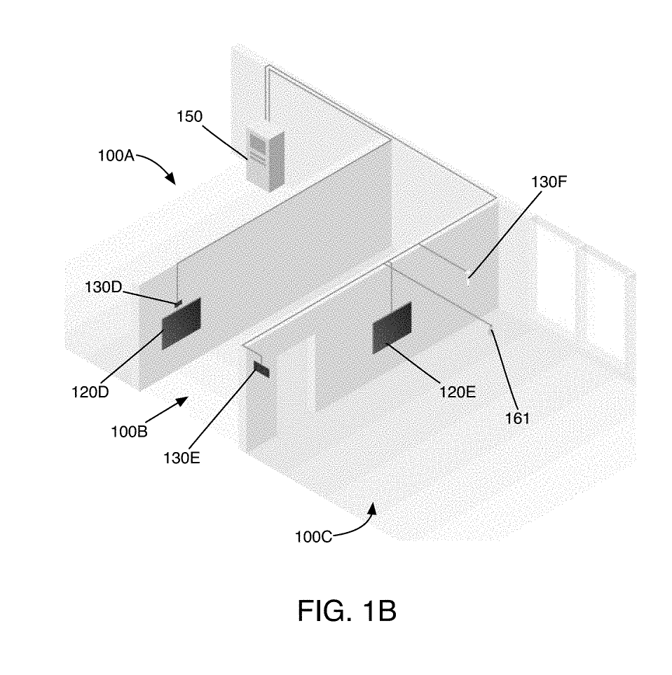

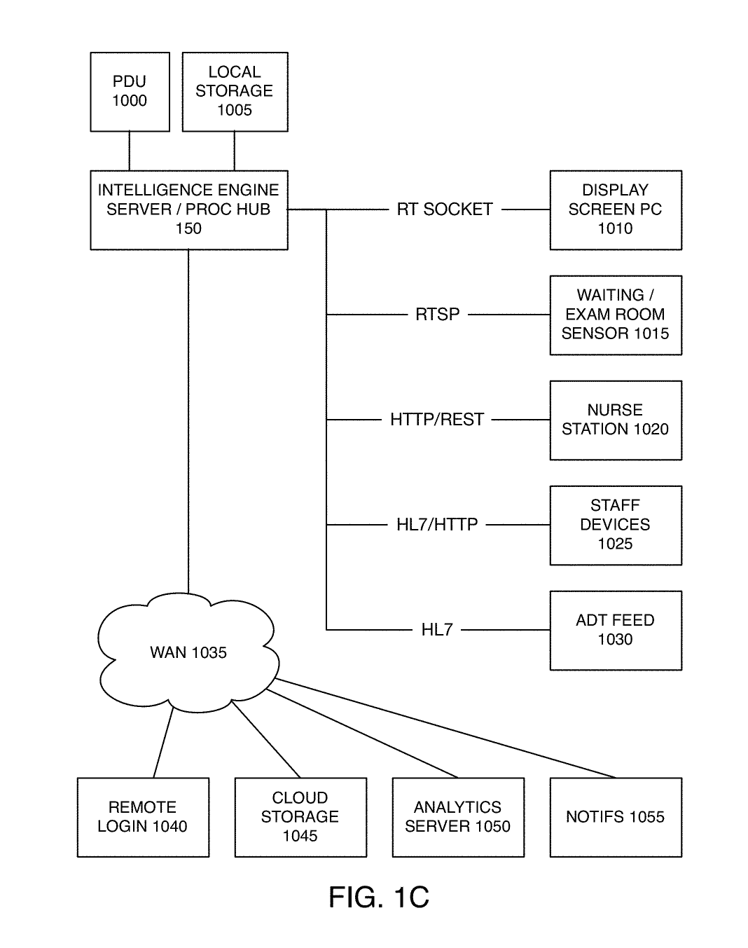

FIG. 1 is a schematic block diagram of a health care facility, in accordance with an exemplary embodiment. Facility 100 includes a local area data network (LAN) 110, which may be a combination of wired and wireless Ethernet networks via which a variety of systems and devices communicate.

Facility interfaces 120 are devices (or combinations of devices) with which occupants of facility 100 may interact, and which may often be publicly-accessible. In some embodiments, an interface 120 may be a display screen, such as a wall-mounted LCD display panel, a matrix of multiple LCD display panels, a projection system, or a personal electronic device (such as a smartphone, tablet computer, smart glasses, smart watch, other wearable devices); the display typically driven by a computer (which may be separate or embedded), for conveying information and media visually to a nearby facility occupant. In some embodiments, an interface 120 may include an audio playback source, which may include a loudspeaker or personal headphones, for conveying audio content to a nearby facility occupant. These and other interfaces may be utilized as a facility interface 120.

Content displayed on an interface 120 may be, for example, a collection of informational, educational, guiding and/or sensory experiences. The color, sound, timing, selection of content and interactions implemented through each interface 120 may be automatically adjusted, as described further herein.

Facility sensors 130 capture information concerning the present state of the facility, and/or people and things within the facility. In some embodiments, a sensor 130 may include a digital video camera system, capturing visual information about various locations within the facility and its occupants. In some embodiments, a sensor 130 may additionally or alternatively include an audio microphone, capturing sounds within facility 100. In some embodiments, sensors 130 may include a short range wireless transceiver adapted for communicating with nearby electronic devices, such as a Bluetooth transceiver or a wireless beacon (which may be implemented using, e.g., the Apple iBeacon and/or Google Eddystone beacon standards). Sensors 130 may be used to observe and/or infer behavioral and physical events taking place within facility 100.

Identification tracking devices 140 are portable wireless devices that may be worn or carried by facility occupants (such as patients or staff). ID tracking devices 140 may interact with facility sensors 130 which include wireless transceivers and may be placed through facility 100 at known locations, in order to identify the current location and identity of individuals within facility 100. In some embodiments, identity tracking device 140 and/or facility sensor 130 may implement a wireless beacon (e.g. using Apple iBeacon and/or Google Eddystone standards) in order to provide indoor location tracking functionality.

In some embodiments, identity tracking devices 140 may further measure and transmit body metrics to a sensor 130 and/or intelligence engine 150 (described further below). Such body metrics may include, e.g., perspiration and heart rate to analyze stress or confusion, amongst other metrics. For example, in some embodiments, identity tracking device 140 may be implemented via a technology wearable having biometric sensors (e.g. heart rate sensors) and one or more wireless data transceivers, such as a smart watch or a smart ring.

Facility interfaces 120 and sensors 130 may communicate with various other devices and computing systems within facility 100, including intelligence engine server 150 and database 155. Database 155 stores information about facility 100 and information related to previous interactions between facility occupants and, e.g., interfaces 120 and sensors 130.

Intelligence engine server 150 is a secure, centralized system that acts as a processing hub. Intelligence engine server 150 keeps track of existing interactions between facility occupants and, e.g., interfaces 120 and sensors 130, as well as behavioral feedback, in order to affect interactive software content. Intelligence engine server 150 profiles and generates categories of events and behavior in order to analyze and make future predictions/preventions. In various embodiments, intelligence engine server 150 may be installed within facility 100, in a separate location, or on the cloud.

Facility 100 may also include one or more facility control systems 160. Many hospitals are outfitted with facility control systems 160 that allow automatic control of the physical space. These can include lighting, sound, shade, temperature setting, real-time location tracking systems, personal health trackers etc. Integrating with these systems allows a facility to connect patients to these systems in a natural way (such as via vocal interactions), while allowing intelligence engine 150 to develop a better understanding of the preferences of patients that stay long-term.

In some embodiments, systems may also integrate with third party identity systems 170 that may be used within a facility 100. Identity system 170 may include, e.g., a key card swipe or RFID badge system. Various levels of integration may be utilized, in order to tie various infrastructure interactions, and presentation of information, to the identity of a person within a facility.

FIG. 1B is a perspective view of an exemplary portion of a facility implementing the system of FIG. 1A. Intelligence engine server 150 is located within a secure facility location 100A, such as a data server room. Public hallway 100B implements a public signage solution that includes display panel 120D and video sensor 130D. Meanwhile, patient room 100C includes video monitoring sensor 130E, patient room display panel 120E, room microphone 130F, and controlled room lighting 161.

FIG. 1C provides an alternative schematic block diagram of a system for health care facility monitoring and occupant interaction, with further illustration of data flow architecture amongst components. The embodiment of FIG. 1C utilizes a combination of local components implemented within the health care facility, as well as cloud-connected components. With regard to local components, intelligence engine server 150, which may also be referred to as processing hub 150, is interconnected with local power distribution unit 1000 and encrypted local storage 1005. Via a facility Ethernet network, server 150 interacts with facility interfaces comprising display screen personal computers 1010, via a real time socket communication protocol. Server 150 interacts with sensors 1015 in waiting and examination rooms using a Real Time Streaming Protocol (RTSP). In some embodiments, content observed by sensors 1015 may be encrypted or otherwise encoded by sensors 1015, prior to streaming to server 150 and/or persistent storage, thereby minimizing risk of inadvertent exposure of private content. Server 150 interacts with nurse station computers 1020 using an HTTP protocol, optionally including implementation of RESTful APIs. Server 150 interacts with staff devices, such as smartphones or tablets, using HTTP protocols, preferably following HL7 standards. Server 150 interacts with ADT feed 1030 in accordance with HL7 standards. Optional cloud-connected components of the system of FIG. 1C interact with server 150 via wide area network 1035, and include remote login server 1040, cloud storage service 1045, analytics service 1050 and notification service 1055.

The health care facility computing environment of FIGS. 1A, 1B and 1C may be employed to infer physical and behavioral events from data captured and conveyed to intelligence engine 150 from sensors 130, facility control system 160 and/or identity system 170. Exemplary operations that may be implemented include, without limitation:

Personalized Wayfindinq within a Facility.

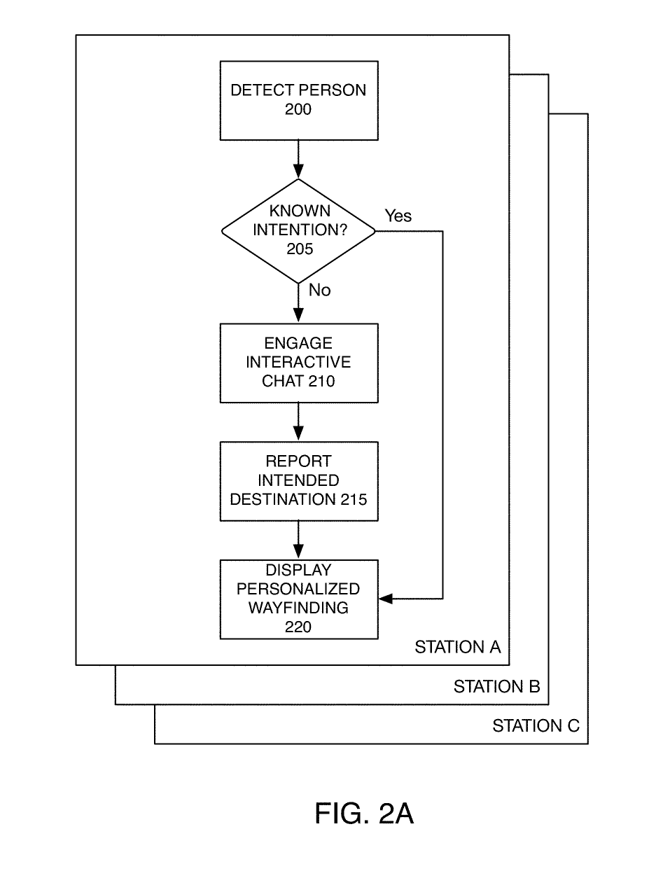

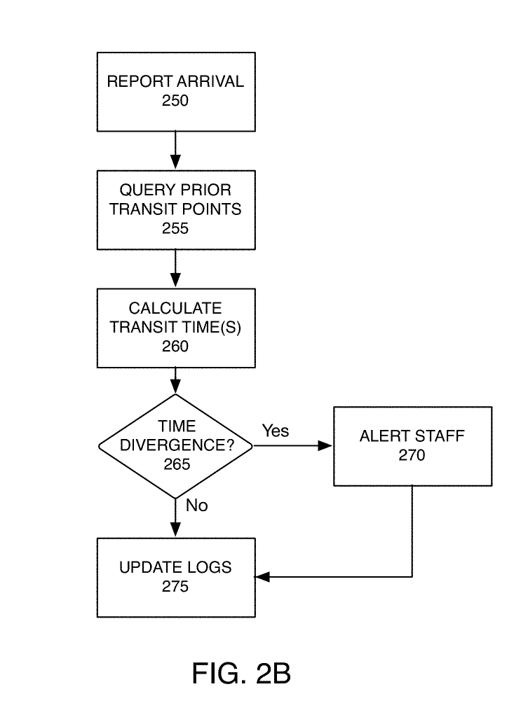

Some embodiments may be utilized for providing patients with personalized, highly-automated wayfinding within a health care facility. FIG. 2A illustrates an exemplary process for such an implementation. In such an embodiment, sensors 130 may include a video camera. The camera, in conjunction with a computer-implemented video processor implementing image and/or video analysis software, detects a person entering the video frame (step 200). In step 205, intelligence engine 150 receives notification of the detected person from the sensor 130, and determines whether the person's identity and intended destination are already known. If not, a chat bot intelligent agent (which may be speech-based, text-based or both) is implemented via a combination of facility interfaces 120 (such as a digital display and loudspeaker) and sensors 130 (such as a microphone and video camera), driven by a chat bot software component operating on, e.g., intelligence engine 150, another cloud-based computing resource, and/or a computing resource local to the display 120. The chat bot intelligent agent engages the new person in a conversation (step 210). Through that conversation, the chat bot agent learns of the user's next intended destination within facility 100 (e.g. the gastroenterology department). The chat bot intelligent agent reports the user's identity and intention to intelligence engine 150 (step 215). The active facility interface 120 then displays personalized wayfinding content for the individual before it, advising the detected occupant of how to get to their intended destination (step 220). The occupant may then move on to a different location within facility 100, whereupon another set of interface 120 and sensors 130 (i.e. STATION B, or STATION C) are encountered and the process is repeated.

However, in step 205, in some circumstances intelligence agent 150 may determine that the user's current intention is already known, such that display 120 will be driven to immediately personalized wayfinding in step 220 (e.g. display directions to the user's intended destination) upon detecting a known individual in steps 200 and 205. The user may then proceed onwards, towards the next display 120 and their destination. Yet, if the user desires to engage with the next display (e.g. due to a change in intended destination), steps 210-220 may be repeated at the new display station. This personalized wayfinding process may be implemented on display/sensor stations distributed throughout a facility to provide comforting, highly-responsive directions to occupants navigating the facility.

Wayfinding Optimization.

Systems described herein having capabilities for patient identification and interaction, may also be utilized for automated optimization of wayfinding within a facility. During wayfinding interactions such as those described in FIG. 2A, patient identity, location and time of presence are determined and stored by intelligence engine server 150 (e.g. within database 155). This information can subsequently be evaluated to determine patient transit times and routes. Deviations in expected transit time or route may then be utilized to alert facility staff of, e.g., common navigational challenges and opportunities for improving directions.

An example process for wayfinding optimization is illustrated in FIG. 2B. For example, a camera sensor 130, operating as described elsewhere herein, recognizes a person (such as using a facial recognition component implemented by intelligence engine server 150 to determine a unique patient identifier), previously seen at the facility lobby, enter the waiting room in the facility's gastroenterology department, and reports the patient's presence and location to intelligence engine 150 (step 250). Intelligence engine 150 connects to database 155 to query the patient's prior transit points (e.g. prior locations and time at location) (step 255), and then determines the time it took for the patient to arrive (step 260). The patient transit time (individually and/or in aggregate with other patients traveling between the lobby and gastroenterology department) may be reported to the staff. Intelligence engine 150 may also evaluate for divergence between actual and expected patient transit times (step 265), and upon identifying a divergence, alert facility staff of the divergence and, for example, recommend more navigations cues to the staff (step 270). Transit logs may also be obtained to facilitate later evaluation (step 275).

Personalized Media Presentation to Optimize Patient Response.

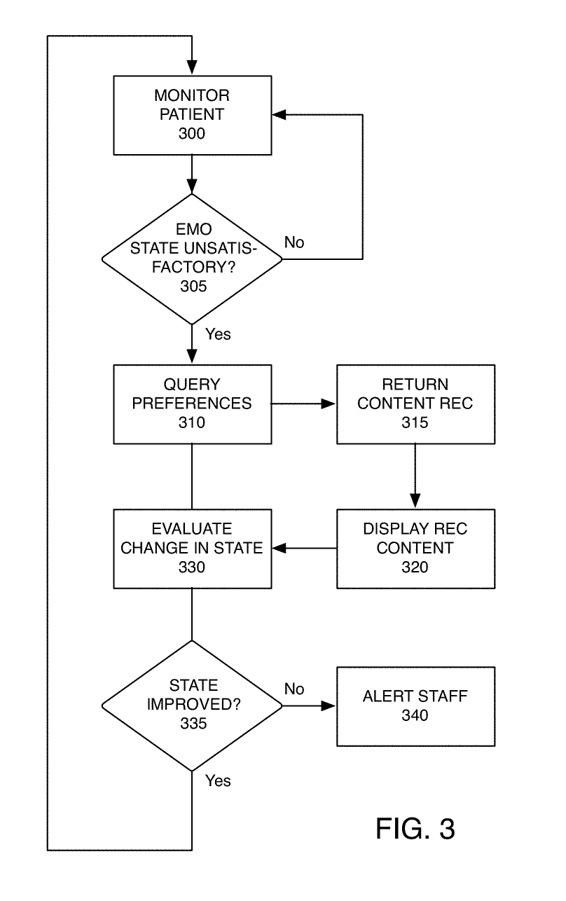

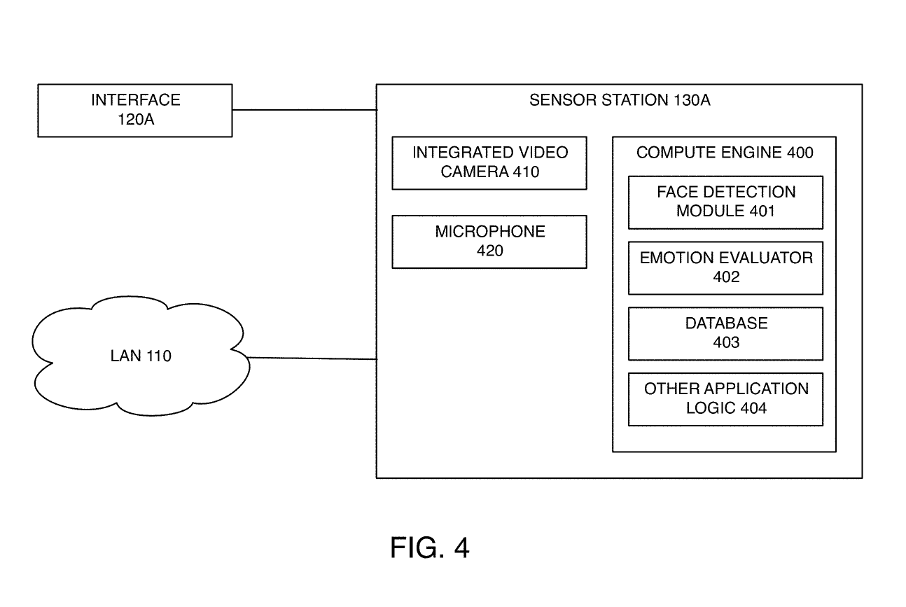

In some embodiments, the satisfaction and happiness of facility occupants may be improved through timely presentation of media content personalized to optimize occupant response. FIG. 3 illustrates an exemplary process, for a facility in which one or more interfaces 120 and sensors 130 are installed within an elderly care facility. FIG. 4 is a schematic block diagram of a sensor and interface package that may be installed within facility 100 for implementing the process of FIG. 3. A patient care room includes interface 120A, connected to integrated sensor station 130A. Integrated sensor station 130A includes sensors, as well as computing resources, such as a small server, preferably implemented in a standalone appliance form factor. Sensor station 130A includes compute engine 400, integrated video camera 410 and microphone 420. Compute engine 400 includes face detection module 401, configured to identify human faces within a video frame. Emotion evaluator 402 applies video analysis logic to video portions identified as faces by module 401, any audio captured by microphone 420, and/or video evaluation of individual posture, to evaluate the emotional state of an individual within the frame of camera 410. Database 403 provides a local store of video, audio and analysis data, and is intended to broadly refer to structured and unstructured stores of data by compute engine 400, whether local, remote or distributed. Compute engine 400 also includes other application logic 404 to communicate with external computing resources, implement a sensor station local user interface, and otherwise carry out functionality described herein.

While depicted in the schematic block diagram of FIG. 4 as a block element with limited sub elements, as known in the art of modern networked computing applications and network services, compute engine 400 (and other servers or computers described herein) may be implemented in a variety of ways, including via distributed hardware and software resources and using any of multiple different software stacks. Computers described herein may include a variety of physical, functional and/or logical components such as one or more each of web servers, application servers, database servers, email servers, SMS or other messaging servers, and the like. That said, the implementation of the servers and other computers will include at some level one or more physical computers, at least one of the physical computers having one or more microprocessors and digital memory for, inter alia, storing instructions which, when executed by the processor, cause the computer to perform methods and operations described herein.

In an exemplary operation, camera 410 monitors an elderly patient within an aged care room, with face detection modules 401 and emotion evaluator 402 processing data received from camera 410 and microphone 420 to evaluate the patient's emotional state (step 300). In step 305, application logic 404 determines whether the emotional state output by evaluator 402 meets criteria for attempting improvement (e.g. illustrating signs of sadness or depression). If not, monitoring may continue (step 300). If so, in step 310, compute engine 400 queries intelligence engine 150 for media content recommendations, based on patient preferences as determined through any prior interactions with the identified patient. Additionally or alternatively, prior responses of other, preferably similarly-situated, patients may be utilized in determining media content recommendations. Content recommendations may be determined utilizing machine learning models for content recommendation, as known in the art, with change in the patient's emotional state upon experiencing the content as a feedback element in determining patient preferences. Other attributes that may be useful in content selection include one or more of, without limitation: patient biographical data, the patient's inferred state prior to media presentation, time of day, and location of media presentation.

In step 315, intelligence engine 150 returns a media content recommendation, personalized for the patient. In step 320, compute engine 400 displays the recommended media content via interface 120A. In step 330, sensor station 130A monitors change in the patient's state upon viewing the content. The change in state is conveyed back to intelligence engine 150, and may be utilized as feedback to the content recommendation component, towards determining future media selections for that patient or others. In step 335, sensor station 130A determines whether the patient's emotional state has improved upon consumption of the presented media. If so, the system returns to monitoring the patient. If not, staff is alerted so that further care may be provided (step 340).

Patient and Facility Monitoring.

Leveraging a distributed network of sensors 130, including video cameras, connected with local or networked image processing components, intelligence engine 150 may monitor for dangerous conditions and alert facility staff.

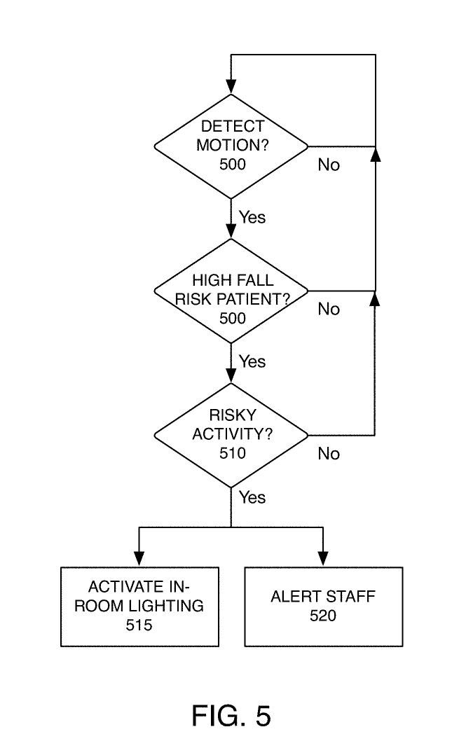

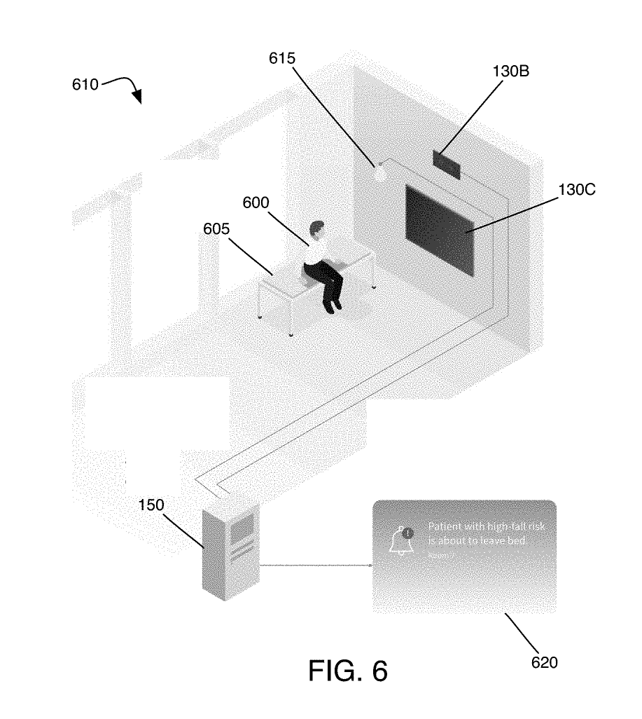

For example, in a patient room, a camera may detect a person who is falling or prone to falling, and alert the staff. FIG. 5 illustrates an exemplary process for mitigating patient fall risk, within a facility such as that illustrated in FIG. 6. In particular, patient 600 is positioned on bed 605 within patient care room 610. Video camera 130B monitors room activity, while digital display 130C provides a mechanism for visual interaction with room occupants. Intelligence engine server 150 receives video information from camera 130B, and transmits data to and from display 130C. Room lighting 615 may be controlled by intelligence engine 150, whether directly or via facility control system 160.

In operation, camera 130B is used to detect motion of room occupants (step 500). If no motion is detecting, monitoring continues. If motion is detected within room 610, intelligence engine server 150 queries room occupant records (whether stored in database 155 or in another network-connected facility data system) to determine whether patient 600 has been identified as a high fall risk. If not, motion detection may continue. If so, intelligence engine 150 may further evaluate whether the detected motion is likely to be activity of the sort having a high fall risk (step 510). For example, a patient in a reclined position, who may be rolling over, may, in some embodiments, be deemed to not constitute a fall risk. In such circumstances, monitoring may continue (e.g. step 500). However, a patient in a reclined position to transitions to an upright seated position, may be considered likely to be preparing to stand, and therefore undertaking an activity having an elevated fall risk. Additionally or alternatively, intelligence engine server 150 may perform image analysis on room occupant video to evaluate joint angles, and identify individuals moving in predetermined ways as having a high fall risk. Further, a video feed may be monitored by intelligence engine 150 to determine that a patient has fallen, such as via rapid movements downwards towards a floor surface.

Intelligence server 150 may then undertake one or more responsive actions, typically intended to mitigate fall risk and/or alert staff to a fall. For example, intelligence server 150 may activate in room lighting 615 (particularly at night or in low light conditions) in order to allow the patient to better perceive their immediate environment prior to further motion (step 515). Additionally or alternatively, intelligence engine 150 may alert staff (such as by transmitting alert message 620 to a nurse station computer display) that a patient is expected to undertake a high fall risk activity or that a patient has already fallen, such that staff monitoring and/or assistance may be provided promptly (step 520). In yet other circumstances, digital display 130C may be driven by intelligence server 150 to display a communication to patient 600, encouraging avoidance of high-fall risk activities until facility staff are present to assist.

Video analysis of patient activity types, mobility, physical condition and behavior may be utilized by intelligence engine 150 as criteria for a variety of different business rules, notifications, activity logging and other events. Examples of facility occupant conditions that may be detected and utilized for such purposes include walking, sitting, standing, laying, sleeping, active motion, falls, and transitions between such states. Underlying occupant physical conditions and motions may also be used to derive patterns and/or hypotheses about higher-level occupant conditions, such as sleep patterns and assessment of comfort level. Such observations and derivations may be utilized, for example, to trigger automated staff notifications of patient conditions, and suggested responsive actions.

In a facility hallway, a camera may also detect a trip hazard and alert the staff. For example, a camera feed may be applied to a video analysis component to identify new objects left motionless in a hallway. Upon identifying such objects, intelligence engine server 150 may transmit a notification to facility staff, alerting of the nature and location of the potential trip hazard for inspection and remediation.

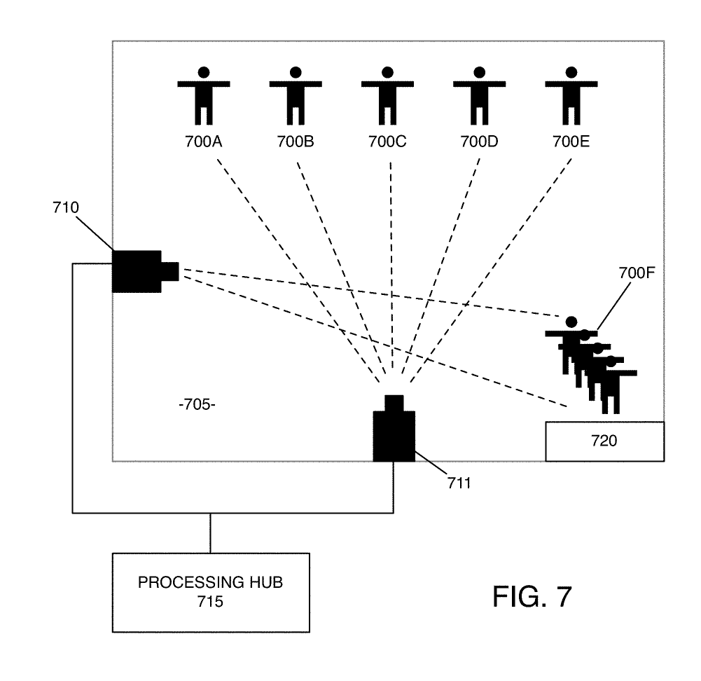

Other facility conditions may also be monitored for alerting and optimization, using the installed network of sensors and compute engines. For example, one or more sensors 130 with video camera components may monitor a facility waiting room. FIG. 7 illustrates such an embodiment. Video camera sensors 710 and 711 monitor individuals within waiting room 705, transmitting collected video data to processing hub 715. Processing hub 715 applies a video analysis component to identify people present, determine whether they are sitting or standing, and/or track the duration of time spent by each person in the waiting room. For example, video camera 711 may observer individuals 700A-E towards providing processing hub 715 with image data that can be processed to differentiate unique individuals, and tally the amount of time each person is waiting in room 705. If processing hub 715 determines that a particular patient has been waiting more than a threshold length of time, facility staff may be alerted to attend to the patient, whether expediting service or providing updated information about expected wait time. The threshold length of time may be determined in a variety of ways. In some embodiments, a fixed wait time threshold may be applied. In some embodiments, where a specific patient can be identified and processing hub 715 can query an appointment time and/or wait time expected at check-in, the threshold for facility staff notification may be determined based on a wait time relative to the expected time and/or appointment time. These and other criteria may be utilized in monitoring patient wait times and initiating staff notifications based thereon.

In some embodiments, video feed from camera 711 may be utilized by processing hub 715 to assess a number of empty chairs in a waiting area and/or a number of people standing. In the event that no further chairs are available and/or a threshold number of people are waiting while standing, processing hub 715 may notify facility staff to bring additional seating and/or take other action to ameliorate potentially uncomfortable waiting conditions.

In some embodiments, a video feed may be utilized to automatically monitor the number of individuals queued at a reception desk, towards notifying staff if additional resources should be deployed to reduce wait time. For example, camera 710 may monitor a queue of individuals 700F, waiting at reception station 720. Video content from camera 710 may be processed by processing hub 715 to trigger staff notifications if patient queue 700F exceeds a threshold number of people.

The individual presence and wait time monitoring described in a waiting room context with regard to FIG. 7, may also be applied in an examination room context. For example, a processing hub or intelligence engine server may monitor exam room occupancy, and patient wait times in an exam room, towards prompting patient interaction and care, providing real-time insight into exam room availability, and optimizing metrics such as average exam time.

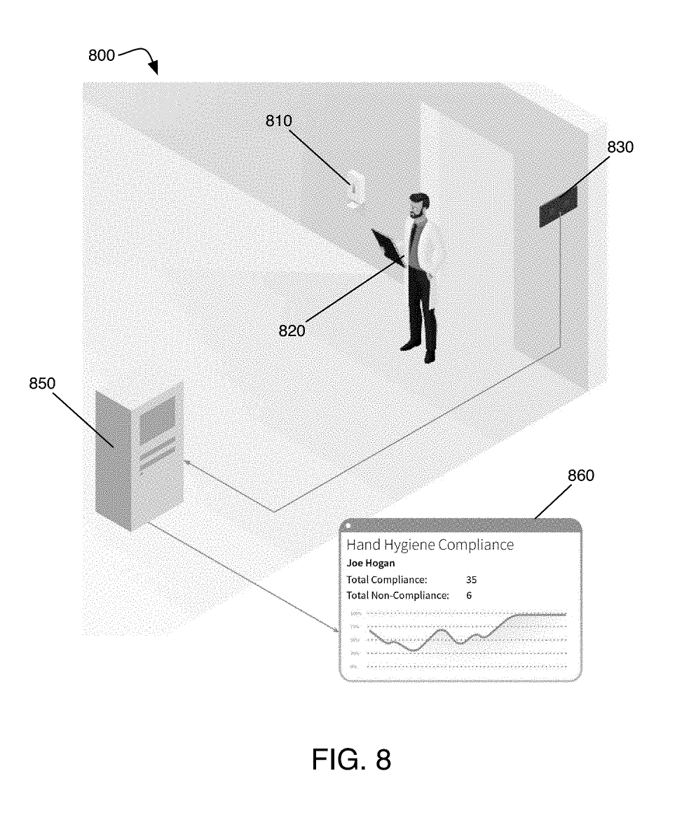

Another application for exam room tracking is hand hygiene compliance. Health care facilities increasingly install hand sanitizing stations within each exam room, with policies requiring staff to take hand sanitizing measures upon each room entry, thereby minimizing risk of cross-contamination between patients, equipment and rooms. FIG. 8 illustrates an implementation of a facility monitoring system, as described herein, to enable automated tracking of staff compliance with hand hygiene policies. Exam room 800 includes hand sanitizing station 810, and video camera sensor 830 providing a video feed to processing hub 850. As a staff member 820 enters exam room 800, video camera sensor 830 monitors the movement of staff member 820 to determine whether staff member 820 utilizes hand sanitizing station 810 (e.g., by detecting movements to the station location, followed by a pause in movement at the station). In some embodiments, the identity of staff member 820 may be determined by video recognition (e.g. by processing hub 850 performing image recognition on a feed from video camera sensor 830). In some embodiments, the identity of staff member 820 may be determined by other means, such as an ID tracking device 140 and/or querying identity system 170, as illustrated in the embodiment of FIG. 1A. When the identity of staff member 820 is determined, processing hub 850 may maintain hand hygiene compliance logs or records on a per-staff-member basis, which compliance can subsequently be queried and used to generate, e.g., compliance report 860. Individualized compliance metrics may be useful in helping staff members develop desired hand hygiene habits. However, even in embodiments for which individual identification cannot be determined, processing hub 850 can track overall metrics concerning hand hygiene compliance rates by individuals entering an exam room, thereby enabling facility administrators to assess overall compliance rates and, for example, measure trends in compliance following training efforts. Furthermore, in some embodiments, in the absence of a detected staff member interaction with a hand hygiene compliance station, processing hub 850 may initiate a hand hygiene reminder, such as a visual reminder rendered by an examination room digital display facility interface 120, and/or a notification initiated by processing hub 130 and transmitted to a staff device (e.g. staff devices 1025).

While embodiments described herein may be beneficially applied to evaluate, track and respond to individual occupants of a health care facility, the results of such systems may also be utilized to generate comprehensive, facility-wide metrics, potentially providing actionable insights for facility improvement.

In addition to tracking patients and staff, some embodiments may also deploy image recognition components to track equipment, thereby enabling intelligence server 150 to provide centralized reporting of equipment location and minimizing opportunities for lost or misplaced equipment.

These and other solutions may be beneficially implemented using the systems and methods for patient monitoring and interaction described herein.

Implementation Across Multiple Facilities.

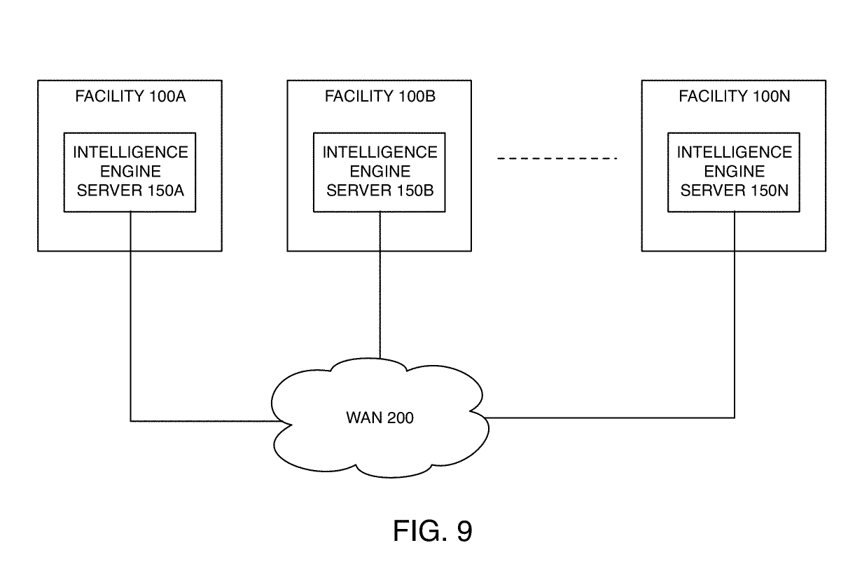

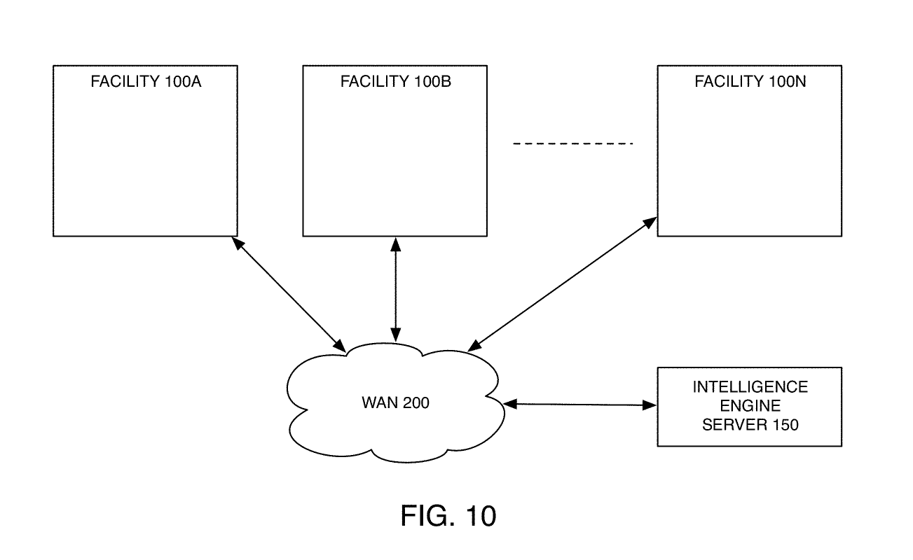

In some embodiments, it may be desirable to implement systems as described herein, across multiple related facilities. FIG. 9 illustrates one such embodiment, with multiple facilities 100A through 100N each having an intelligence server 150A through 150N, respectively. Intelligence servers 150 communication via wide area network 200. By enabling interaction between multiple intelligence servers at related facilities, learnings and optimizations may be applied across an entire collection of related facilities and those facilities' patients, potentially improving the rapidity and extent to which operations may be optimized. FIG. 10 illustrates an alternative multi-facility embodiment, in which a centralized, cloud-based intelligence engine 150 communicates with multiple facilities 100A through 100N, via WAN 200. It is contemplated and understood that in yet other embodiments, other variations on compute engine topology may be desirable. For example, intelligence engine duties may be distributed between local, facility-specific intelligence engine servers and a centralized intelligence engine server. In yet other embodiments, it may be desirable to install a local intelligence engine server within large facilities, such as a hospital, while affiliated small facilities (such as a local clinic) may rely on a cloud-based intelligence engine server.

While certain embodiments of the invention have been described herein in detail for purposes of clarity and understanding, the foregoing description and Figures merely explain and illustrate the present invention and the present invention is not limited thereto. It will be appreciated that those skilled in the art, having the present disclosure before them, will be able to make modifications and variations to that disclosed herein without departing from the scope of any appended claims.

1. A method for personalized wayfinding in a health care facility comprising:
identifying a patient at a first of a plurality of wayfinding stations distributed at known locations within the health care facility, the wayfinding stations each comprising a digital display screen, a video camera sensor, and a compute engine;
querying a central data repository to identify an intended destination associated with the identified patient; and
displaying, on the digital display screen, wayfinding instructions directing the patient from the known location of the first wayfinding station, to the intended destination.

2. The method of claim 1, in which the step of identifying a patient comprises:
capturing one or more images of the patient approaching the first wayfinding station; and
applying the one or more captured images to query a facial recognition component, the facial recognition component returning a patient identification.

3. The method of claim 2, in which the facial recognition component is implemented on a centralized facility intelligence server communicating with the one or more wayfinding stations via a local area network.

4. The method of claim 1, in which the wayfinding stations further comprise a microphone and loudspeaker, and in which the step of querying a central data repository to identify an intended destination associated with the identified patient further comprises:
receiving, by the first wayfinding station, an indication that the patient's intended destination is unknown; and
querying the patient, by the first wayfinding station, for an intended destination, through implementation of an audio chat agent at least in part using the first wayfinding station compute engine.

5. The method of claim 1, further comprising:
reporting, by a destination wayfinding station to a central intelligence engine server, that the patient has arrived at the intended destination;
determining, by the central intelligence engine server, actual transit times for the patient; and
transmitting notification to facility staff of divergence between actual transit times for the patient and expected transit times.

6. A method for monitoring patient satisfaction in a health care facility waiting area, the method comprising:
identifying each of one or more patients in the waiting area by:
(a) transmitting a plurality of images, each captured at a known time, from one or more video cameras directed towards the waiting area, to a processing hub implementing application logic comprising an image recognition component;
(b) applying the image recognition component by the processing hub to uniquely identify each of the one or more patients in each of the images;
tracking, by the processing hub, a waiting duration of time during which each of the patients is present in the waiting area; and
transmitting a notification to facility staff in the event that waiting duration for a patient has exceeded a threshold level.

7. The method of claim 6, in which the waiting duration threshold level is predetermined.

8. The method of claim 6, in which the waiting duration threshold level is determined by comparison of a current time to a patient appointment time, the patient appointment time determined by querying a compute server implementing a patient scheduling service.

9. The method of claim 6, further comprising:
applying the one or more images to an emotion evaluator module implemented by the processing hub application logic;
determining that one or more of the patients is illustrating signs of an unsatisfactory emotional state; and
transmitting, by the processing hub, a notification to a network-connected computing device associated with facility staff, identifying the one or more patients illustrating signs of an unsatisfactory emotional state.

10. The method of claim 6, further comprising:
transmitting a plurality of images, each captured at a known time, from a video camera directed towards a reception station;
applying an image analysis component implemented by the processing hub application logic to determine a number of individuals queued at the reception station; and
initiating, by the processing hub, transmission of a notification to a network-connected computing device associated with facility staff indicating that the number of individuals queued at the reception station has exceeded a threshold level.

11. A system for monitoring and interacting with occupants of a health care facility comprising:
one or more video sensors distributed throughout a health care facility at known locations, each video sensor streaming observed content to a processing hub via a local area network;
the processing hub comprises application logic implementing a video content analysis module to observed content received from the one or more video sensors, the video content analysis module determining, for each of one or more individuals, an individual identity and a state or activity associated with the individual; and
one or more facility staff terminals receiving notifications of identity, location and state or activity associated with one or more of the individuals.

12. The system of claim 11, in which the video content analysis module comprises application logic for tracking hand hygiene compliance.

13. The system of claim 11, in which the video content analysis module comprises application logic for tracking patient emotional state.

14. The system of claim 11, in which the video content analysis module comprises application logic for tracking patient waiting room time.

15. A method for monitoring hand hygiene compliance in a health care facility, the method comprising:
transmitting a video feed from a video camera sensor installed in an examination room to a processing hub, the video camera sensor directed towards a hand hygiene station;
identifying a facility staff member upon entry to the examination room;
applying content from the video feed to an image analysis component implemented by processing hub application logic, the image analysis component configured to detect staff member interaction with the hand hygiene station and generate hand hygiene compliance logs; and
transmitting a hand hygiene compliance report comprising information from the hand hygiene compliance logs.

16. The method of claim 15, in which the step of identifying a staff member upon entry to the examination room comprises:
capturing one or more images of the staff member upon entry to the examination room by the video camera sensor;
applying the one or more images of the staff member to query a facial recognition component implemented by processing hub application logic.

17. The method of claim 15, in which the step of identifying a staff member upon entry to the examination room comprises querying a health care facility identity system.

18. The method of claim 15, further comprising:
in the absence of detecting a staff member interaction with the hand hygiene station, initiating, by the processing hub, display of a compliance reminder on an examination room digital display.
